SEQUENCE LISTING (48 sequences)

SEQ ID NO: 1 - protein, 11 aa, synthetic construct
KASQNVGTNVA

SEQ ID NO: 2 - protein, 7 aa, synthetic construct
SASYRYS

SEQ ID NO: 3 - protein, 8 aa, synthetic construct
QQYNSYYT

SEQ ID NO: 4 - protein, 8 aa, synthetic construct
QQYSSYYT

SEQ ID NO: 5 - protein, 10 aa, synthetic construct
GYTFTNYVMH

SEQ ID NO: 6 - protein, 17 aa, synthetic construct
YILPYNDLTKYNEKFKG

SEQ ID NO: 7 - protein, 10 aa, synthetic construct
HDYDGGVMDY

SEQ ID NO: 8 - protein, 10 aa, synthetic construct
HDYDAGVMDY

SEQ ID NO: 9 - protein, 23 aa, synthetic construct
DIVLTQSQKFMSTSVGDRVSVTC

SEQ ID NO: 10 - protein, 23 aa, synthetic construct
DIQLTQSPSTLSASVGDRVTITC

SEQ ID NO: 11 - protein, 15 aa, synthetic construct
WYQQKPGQSPKALIY

SEQ ID NO: 12 - protein, 15 aa, synthetic construct
WYQQKPGKAPKALIY

SEQ ID NO: 13 - protein, 32 aa, synthetic construct
GVPDRFTGSGSGTDFTLTISNVQSEDLAEYFC

SEQ ID NO: 14 - protein, 32 aa, synthetic construct
GVPSRFSGSGSGTEFTLTISSLQPDDFATYYC

SEQ ID NO: 15 - protein, 12 aa, synthetic construct
FGGGTKLELKRT

SEQ ID NO: 16 - protein, 12 aa, synthetic construct
FGQGTRLEIKGG

SEQ ID NO: 17 - protein, 25 aa, synthetic construct
EVQLQQSGPELVKPGASVKMSCKAS

SEQ ID NO: 18 - protein, 25 aa, synthetic construct [sequence omitted]

SEQ ID NO: 19 - protein, 14 aa, synthetic construct
WVKQKPGQGLEWIG

SEQ ID NO: 20 - protein, 14 aa, synthetic construct
WVRQAPGQGLEWIG

SEQ ID NO: 21 - protein, 14 aa, synthetic construct
WVRQAPGQGLEWMG

SEQ ID NO: 22 - protein, 32 aa, synthetic construct
KATLSSDRSSSTAYMELSSLTSEDSAVYYCAR

SEQ ID NO: 23 - protein, 14 aa, synthetic construct
WVRQAPGQGLEWIG

SEQ ID NO: 24 - protein, 32 aa, synthetic construct
RVTLTSDRSTSTVYMELSSLRSEDTAVYYCAR

SEQ ID NO: 25 - protein, 11 aa, synthetic construct
WGQGTSVTVSS

SEQ ID NO: 26 - protein, 11 aa, synthetic construct
WGQGTTVTVSS

SEQ ID NO: 27 - protein, 242 aa, synthetic construct
DIVLTQSQKFMSTSVGDRVSVTCKASQNVGTNVAWYQQKPGQSPKALIYSASYRYSGVPDRFTGSGSGTDFTLTISNVQSEDLAEYFCQQYNSYYTFGGGTKLELKRTGGGGSGGGGSGGGGSEVQLQQSGPELVKPGASVKMSCKASGYTFTNYVMHWVKQKPGQGLEWIGYILPYNDLTKYNEKFKGKATLSSDRSSSTAYMELSSLTSEDSAVYYCARHDYDGGVMDYWGQGTSVTVSS

SEQ ID NO: 28 - protein, 242 aa, synthetic construct; identical to SEQ ID NO: 27

SEQ ID NO: 29 - protein, 240 aa, synthetic construct
DIQLTQSPSTLSASVGDRVTITCKASQNVGTNVAWYQQKPGQSPKALIYSASYRYSGVPSRFSGSGSGTEFTLTISSLQPDDFATYYCQQYNSYYTFGQGTRLEIKGGGGSGGGGSGGGGSQVQLVQSGAEVKKPGASVKVSCKASGYTFTNYVMHWVRQAPGQGLEWIGYILPYNDLTKYNEKFKGRATLTSDRSTSTAYMELSSLRSEDTAVYYCARHDYDGGVMDYWGQGTTVTVSS

SEQ ID NO: 30 - protein, 240 aa, synthetic construct
DIQLTQSPSTLSASVGDRVTITCKASQNVGTNVAWYQQKPGQSPKALIYSASYRYSGVPSRFSGSGSGTEFTLTISSLQPDDFATYYCQQYSSYYTFGQGTRLEIKGGGGSGGGGSGGGGSQVQLVQSGAEVKKPGASVKVSCKASGYTFTNYVMHWVRQAPGQGLEWIGYILPYNDLTKYNEKFKGRATLTSDRSTSTAYMELSSLRSEDTAVYYCARHDYDGGVMDYWGQGTTVTVSS

SEQ ID NO: 31 - protein, 240 aa, synthetic construct
DIQLTQSPSTLSASVGDRVTITCKASQNVGTNVAWYQQKPGQSPKALIYSASYRYSGVPSRFSGSGSGTEFTLTISSLQPDDFATYYCQQYNSYYTFGQGTRLEIKGGGGSGGGGSGGGGSQVQLVQSGAEVKKPGASVKVSCKASGYTFTNYVMHWVRQAPGQGLEWIGYILPYNDLTKYNEKFKGRATLTSDRSTSTAYMELSSLRSEDTAVYYCARHDYDAGVMDYWGQGTTVTVSS

SEQ ID NO: 32 - protein, 240 aa, synthetic construct
DIQLTQSPSTLSASVGDRVTITCKASQNVGTNVAWYQQKPGQSPKALIYSASYRYSGVPSRFSGSGSGTEFTLTISSLQPDDFATYYCQQYSSYYTFGQGTRLEIKGGGGSGGGGSGGGGSQVQLVQSGAEVKKPGASVKVSCKASGYTFTNYVMHWVRQAPGQGLEWIGYILPYNDLTKYNEKFKGRATLTSDRSTSTAYMELSSLRSEDTAVYYCARHDYDAGVMDYWGQGTTVTVSS

SEQ ID NO: 33 - protein, 240 aa, synthetic construct
DIQLTQSPSTLSASVGDRVTITCKASQNVGTNVAWYQQKPGKAPKALIYSASYRYSGVPSRFSGSGSGTEFTLTISSLQPDDFATYYCQQYSSYYTFGQGTRLEIKGGGGSGGGGSGGGGSQVQLVQSGAEVKKPGASVKVSCKASGYTFTNYVMHWVRQAPGQGLEWMGYILPYNDLTKYNEKFKGRVTLTSDRSTSTVYMELSSLRSEDTAVYYCARHDYDGGVMDYWGQGTTVTVSS

SEQ ID NO: 34 - protein, 240 aa, synthetic construct
DIQLTQSPSTLSASVGDRVTITCKASQNVGTNVAWYQQKPGKAPKALIYSASYRYSGVPSRFSGSGSGTEFTLTISSLQPDDFATYYCQQYNSYYTFGQGTRLEIKGGGGSGGGGSGGGGSQVQLVQSGAEVKKPGASVKVSCKASGYTFTNYVMHWVRQAPGQGLEWMGYILPYNDLTKYNEKFKGRVTLTSDRSTSTVYMELSSLRSEDTAVYYCARHDYDAGVMDYWGQGTTVTVSS

SEQ ID NO: 35 - protein, 240 aa, synthetic construct
DIQLTQSPSTLSASVGDRVTITCKASQNVGTNVAWYQQKPGKAPKALIYSASYRYSGVPSRFSGSGSGTEFTLTISSLQPDDFATYYCQQYSSYYTFGQGTRLEIKGGGGSGGGGSGGGGSQVQLVQSGAEVKKPGASVKVSCKASGYTFTNYVMHWVRQAPGQGLEWMGYILPYNDLTKYNEKFKGRVTLTSDRSTSTVYMELSSLRSEDTAVYYCARHDYDAGVMDYWGQGTTVTVSS

SEQ ID NO: 36 - DNA, 726 bp, synthetic construct
  source 1..726: mol_type = other DNA
gacattgtgctgacccaatctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagaatatttctgtcagcaatataacagctattacacgttcggaggggggaccaagctggagctgaaacgtacgggtggtggcggctcgggcggcggcgggtcgggaggaggaggatccgaggtccagctgcaacaatctggacctgagctggtaaagcctggggcttcagtgaagatgtcctgcaaggcttctggatacacattcactaactatgttatgcactgggtgaagcagaagcctgggcagggccttgagtggattggatatattcttccttacaatgatcttactaagtacaatgagaaattcaaaggcaaggccacactgtcttcagacagatcctccagcacagcctacatggagctcagcagcctgacctctgaggactctgcggtctattactgtgcaagacatgattacgacggaggtgttatggactactggggtcaaggaacctcagtcacagtctcctca

SEQ ID NO: 37 - DNA, 726 bp, synthetic construct
  source 1..726: mol_type = other DNA
gacatcgtgctgacccagagccagaagttcatgtccaccagcgtgggcgacagagtgagcgtgacatgtaaggccagccagaacgtgggcacaaacgtggcctggtatcaacagaagcccggccagagccctaaggccctgatctacagcgccagctacagatactccggcgtgcctgatagattcaccggcagcggcagcggcaccgacttcaccctgaccatcagcaacgtgcagtccgaggatctggccgagtacttttgtcagcagtacaactcctactacacattcggcggcggcaccaagctggagctgaagaggaccggcggcggcggaagcggaggaggaggatctggcggcggcggttccgaggtgcagctgcaacagagcggccctgagctggtgaagcccggcgctagcgtgaagatgagctgcaaggcctccggctacacattcactaactacgtgatgcactgggtgaagcagaagcctggccagggcctggagtggatcggctacatcctgccttacaacgatctgacaaagtacaacgagaagtttaagggcaaggccacactgtcctccgacagatcctcctccaccgcctacatggagctgtcctccctgacaagcgaggactccgccgtgtactactgcgccaggcacgactacgacggcggcgtgatggactactggggccagggcacaagcgtgacagtgtccagc

SEQ ID NO: 38 - DNA, 720 bp, synthetic construct
  source 1..720: mol_type = other DNA
gacatccagctgacacagagccccagcacactgagcgccagcgtgggcgatagggtgaccatcacatgtaaggcctcccagaacgtgggcaccaacgtggcctggtatcaacagaagcctggccagtcccccaaggccctgatctacagcgccagctacagatacagcggcgtgccctccagattctccggctccggctccggaaccgagttcaccctgacaatcagctccctgcagcccgatgactttgccacctactactgccagcagtacaatagctactacaccttcggccagggcacaaggctggagatcaagggcggcggcggcagcggaggaggaggaagcggaggaggcggcagccaggtgcagctggtgcagagcggcgccgaggtgaagaagcccggcgctagcgtgaaggtgtcctgcaaggcctccggctacacattcaccaactacgtgatgcactgggtgagacaggcccccggccagggactggagtggatcggatacatcctgccctacaacgacctgaccaagtacaacgagaagttcaagggcagagccaccctgaccagcgacaggtccaccagcacagcctacatggagctgtcctccctgagaagcgaggacacagccgtgtactactgtgccaggcacgattacgatggcggcgtgatggattactggggccagggcaccaccgtgaccgtgtccagc

SEQ ID NO: 39 - DNA, 720 bp, synthetic construct
  source 1..720: mol_type = other DNA
gatatccagctgacccagagccctagcacactgtccgccagcgtgggcgatagagtgaccatcacctgtaaggccagccagaacgtgggcacaaatgtggcctggtatcaacagaagcctggccagagccccaaggccctgatctacagcgcctcctacagatactccggcgtgccctccaggtttagcggctccggcagcggcacagagttcacactgacaatctcctccctgcagcccgatgattttgccacctactactgtcagcagtacagcagctactacaccttcggccagggcaccaggctggagatcaagggcggcggcggcagcggaggaggaggaagcggaggaggcggctcccaggtgcagctggtgcagtccggcgccgaggtgaagaagcctggcgcctccgtgaaggtgtcctgcaaggcctccggctacacattcactaactacgtgatgcactgggtgagacaggcccccggccagggactggagtggatcggatacatcctgccctacaacgatctgaccaagtacaatgagaagttcaagggcagggccaccctgacaagcgataggagcacctccaccgcctacatggagctgtccagcctgagaagcgaggataccgccgtgtactactgcgccaggcacgattacgacggcggcgtgatggattactggggccagggcacaaccgtgacagtgagcagc

SEQ ID NO: 40 - DNA, 720 bp, synthetic construct
  source 1..720: mol_type = other DNA
gacatccagctgacccagagcccctccaccctgagcgcctccgtgggagatagagtgacaatcacctgcaaggcctcccagaatgtgggcaccaatgtggcctggtatcaacagaagcccggccagtcccccaaggccctgatctactccgcctcctacagatactccggcgtgccctccagattttccggcagcggcagcggcacagagttcacactgaccatcagctccctgcagcccgacgactttgccacctactactgtcagcagtacaatagctactacacatttggccagggcaccagactggagatcaagggcggcggcggcagcggaggaggaggaagcggaggaggcggcagccaggtgcagctggtgcagagcggcgccgaggtgaagaagcccggcgcttccgtgaaggtgagctgtaaggcctccggctacacattcaccaactacgtgatgcactgggtgagacaggcccctggccagggcctggagtggatcggatacatcctgccttacaacgatctgaccaagtacaacgagaagtttaagggcagggccaccctgacaagcgataggagcacaagcaccgcctacatggagctgtccagcctgagaagcgaggatacagccgtgtactactgtgccagacacgactacgacgccggcgtgatggactactggggccagggcacaacagtgacagtgtccagc

SEQ ID NO: 41 - DNA, 720 bp, synthetic construct
  source 1..720: mol_type = other DNA
gatatccagctgacacagagcccttccaccctgagcgcctccgtgggcgacagagtgaccatcacctgcaaggcctcccagaacgtgggcacaaatgtggcctggtatcaacagaagcccggccagtcccccaaggccctgatctactccgccagctacaggtacagcggcgtgccctccagattctccggcagcggcagcggcacagagtttacactgaccatcagctccctgcagcctgacgacttcgccacctactactgccagcagtacagctcctactacaccttcggccagggcacaagactggagatcaagggcggcggcggcagcggaggaggaggaagcggaggaggcggctcccaggtgcagctggtgcagtccggcgccgaggtgaagaagcctggcgcctccgtgaaggtgagctgtaaggcctccggctacaccttcacaaactacgtgatgcactgggtgaggcaggcccccggccagggactggaatggatcggctacatcctgccctacaacgatctgacaaagtacaatgagaagtttaagggcagagccacactgacatccgatagaagcacatccaccgcctacatggagctgagctccctgaggtccgaggataccgccgtgtactactgtgccagacacgactacgacgccggcgtgatggactactggggccagggcaccaccgtgacagtgtcctcc

SEQ ID NO: 42 - DNA, 720 bp, synthetic construct
  source 1..720: mol_type = other DNA
gacatccagctgacccagagccccagcacactgagcgccagcgtgggcgatagagtgaccatcacatgcaaggccagccagaatgtgggcacaaatgtggcctggtatcaacagaagcccggcaaggcccctaaggccctgatctacagcgccagctacagatactccggcgtgcctagcagattcagcggcagcggctccggcaccgagtttacactgaccatcagcagcctgcagcctgatgattttgccacctactactgccagcagtacagcagctactacaccttcggccagggcaccaggctggagatcaagggcggcggcggcagcggaggaggaggaagcggaggaggcggctcccaggtgcagctggtgcagagcggcgccgaggtgaagaagcccggcgcttccgtgaaggtgtcctgtaaggccagcggctacacctttacaaactacgtgatgcactgggtgaggcaggcccctggccagggactggagtggatgggctacatcctgccctacaatgacctgaccaagtacaacgagaagtttaagggcagagtgacactgacatccgataggtccacatccaccgtgtacatggagctgagctccctgaggagcgaggacaccgccgtgtactactgcgccagacacgactacgatggcggcgtgatggactactggggccagggcacaacagtgaccgtgtcctcc

SEQ ID NO: 43 - DNA, 720 bp, synthetic construct
  source 1..720: mol_type = other DNA
gacatccagctgacacagagcccctccaccctgtccgcctccgtgggagatagggtgaccatcacatgtaaggcctcccagaacgtgggcaccaacgtggcctggtatcaacagaagcccggcaaggcccctaaggccctgatctacagcgccagctacagatactccggcgtgccctccagattttccggctccggcagcggcacagagtttacactgaccatctccagcctgcagcccgacgactttgccacctactactgtcagcagtacaacagctactacacctttggccagggcaccagactggagatcaagggcggcggcggcagcggaggaggaggaagcggaggaggcggctcccaggtgcagctggtgcagtccggcgccgaggtgaagaagcctggcgcctccgtgaaggtgtcctgtaaggccagcggctacacctttacaaactacgtgatgcactgggtgaggcaggcccctggccagggactggagtggatgggctacatcctgccctacaacgacctgacaaagtacaacgagaagttcaagggcagagtgaccctgacaagcgataggtccacctccaccgtgtacatggagctgtccagcctgaggtccgaggataccgccgtgtactactgcgccagacacgactacgatgccggcgtgatggattactggggccagggcacaaccgtgaccgtgtcctcc

SEQ ID NO: 44 - DNA, 720 bp, synthetic construct
  source 1..720: mol_type = other DNA
gatatccagctgacccagtcccccagcacactgagcgccagcgtgggcgatagggtgaccatcacctgcaaggccagccagaacgtgggcaccaacgtggcctggtatcaacagaagcctggcaaggcccccaaggccctgatctactccgccagctacaggtacagcggcgtgcctagcaggttcagcggctccggctccggcacagagttcaccctgaccatctccagcctgcagcctgacgattttgccacctactactgtcagcagtacagcagctactacacattcggccagggcacaagactggagatcaagggcggcggcggcagcggaggaggaggaagcggaggaggcggcagccaggtgcagctggtgcagagcggcgccgaggtgaagaagcctggcgccagcgtgaaggtgagctgtaaggccagcggctacacattcacaaattacgtgatgcactgggtgagacaggcccccggccagggactggagtggatgggatacatcctgccctacaacgacctgacaaagtacaacgagaagtttaagggcagggtgaccctgacatccgacagaagcacctccaccgtgtacatggagctgtcctccctgagatccgaggacacagccgtgtactactgtgccagacacgactacgatgccggcgtgatggattactggggccagggcaccaccgtgaccgtgagctcc

SEQ ID NO: 45 - protein, 21 aa, synthetic construct
MALPVTALLLPLALLLHAARP

SEQ ID NO: 46 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 47 - protein, 42 aa, synthetic construct
KRGRKKLLYIFKQPFMRPVQTTQEEDGCSCRFPEEEEGGCEL

SEQ ID NO: 48 - protein, 112 aa, synthetic construct
RVKFSRSADAPAYKQGQNQLYNELNLGRREEYDVLDKRRGRDPEMGGKPRRKNPQEGLYNELQRDRMAEAYSEIGMKGERRRGKGHDGLYQGLSTATKDTYDALHMQALPPR

(19) United States
(12) Patent Application Publication
(10) Pub. No.: US 2025/0115674 A1
(43) Pub. Date: Apr. 10, 2025

(54) ANTIBODY TARGETING B CELL MATURATION ANTIGEN AND RELATED PRODUCTS THEREOF AND MEDICAL APPLICATIONS

(71) Applicant: Beijing Gene MiNK Biotechnology CO.LTD, Beijing (CN)

(72) Inventors: Zhihua Lu, Beijing (CN); Yongfeng Liu, Beijing (CN)

(21) Appl. No.: 18/901,182

(22) Filed: Sep. 30, 2024

(30) Foreign Application Priority Data
Oct. 8, 2023 (CN) 202311289005.6

(51) Int. Cl. C07K 16/28; A61K 31/203; A61K 31/404; A61K 31/44; A61K 31/506; A61K 31/517; A61K 31/5377; A61K 31/69; A61K 33/00; A61K 39/00; A61K 39/395; A61P 35/00; C07K 14/705; C12N 5/0783; G01N 33/68

(52) U.S. Cl. CPC C07K 16/2878; A61K 31/203; A61K 31/404; A61K 31/44; A61K 31/506; A61K 31/517; A61K 31/5377; A61K 31/69; A61K 33/00; A61K 39/3955; A61K 40/15; A61K 40/31; A61K 40/4202; A61P 35/00; C07K 14/70517; C07K 14/70578; C12N 5/0636; G01N 33/6854; C07K 2317/24; C07K 2317/53; C07K 2317/92; C07K 2319/02; C07K 2319/03; C12N 2510/00; G01N 2333/70578

(57) ABSTRACT

The present disclosure belongs to the fields of biotechnology and biological medicine, and specifically relates to an antibody targeting a B cell maturation antigen (BCMA) and related products thereof and medical applications. Specifically, the antibody contains a variable light chain and a variable heavy chain. The variable light chain contains a light chain complementarity-determining region (CDR) 1, a light chain CDR2, and a light chain CDR3. The light chain CDR1 has a sequence as shown in SEQ ID NO. 1, the light chain CDR2 has an amino acid sequence as shown in SEQ ID NO. 2, and the light chain CDR3 is as shown in SEQ ID NO. 3 or SEQ ID NO. 4. Chimeric antigen receptor-natural killer (CAR-NK) cells prepared by a humanized antibody provided in the present disclosure are high in stability of positive rate and in killing capacity.

### SEQUENCE LISTING

The instant application contains a Sequence Listing with 48 sequence, which has been submitted electronically in XML format and is hereby incorporated herein by reference in its entirety. Said XML copy, created on Sep. 24, 2024, is named HBJS-US-1-30-SEQ.xml, and is 50.1 kbytes in size.

### CROSS-REFERENCE TO RELATED APPLICATION

This application claims priority of Chinese Patent Application No. 202311289005.6, filed on Oct. 8, 2023, the entire contents of which are incorporated herein by reference.

### TECHNICAL FIELD

The present disclosure belongs to the fields of biotechnology and biological medicine, and specifically relates to an antibody targeting a B cell maturation antigen (BCMA) and related products thereof and medical applications.

### BACKGROUND

BCMA, i.e. CD269, a member of tumor necrosis factor (TNF) receptor super-family, can bind to B cell activating factors and proliferation-inducing ligands, and is preferentially expressed in mature B lymphocytes. BCMA is expressed significantly higher in multiple myeloma (MM) than in healthy plasma cells, and a growing number of evidences show that BCMA acts as one of the target antigens suitable for treating MM.

Limited by ethics, experimental animals including mice, rabbits and alpacas, rather than human body are served for antibody screening, of which, mice are most extensively applied due to minimum screening cost, and accordingly, most antibodies are mouse-derived. As monoclonal drugs emerge, followed by chimeric antigen receptor T (CAR-T) and CAR-natural killer (CAR-NK) cell immunotherapies, the antibody screening based on experimental animals is further applied in an extensive field over recent years. Following the extensive application of products based on monoclonal antibody, CAR-T and CAR-NK cells, researches find that if the animal-derived antibodies such as mouse-derived antibodies, and CAR are directly applied in human body, they are regarded as heterogeneous and heterologous proteins by an immune system of human body. This allows for the generation of obvious antigen-antibody reaction, resulting in a lot of antibodies being produced in human body, thereby affecting the therapeutic effect of mouse-derived antibodies on the human body. To solve this problem, one of the effective approaches involves the usage of bioinformatics data and knowledge to optimize codons of nucleic acid sequences of the screened mouse-derived antibodies, allowing the antibody to be expressed in more consistent with the expression habits in the human body. Meanwhile, bioinformatics algorithms are employed to perform humanized optimization on framework regions (FRs) of the antibodies in the case that the complementarity-determining region (CDR) remains unchanged, so that the sequences of the mouse-derived antibodies are ultimately transformed to sequences close to sequences of natural antibodies in human body to the greatest extent. The optimized antibodies, which are known as humanized antibodies, show no significant antigen-antibody reactions in human body, maximizing the therapeutic effect of the antibody drugs, CAR-T, and CAR-NK.

### SUMMARY

In the present disclosure, a sequence of a mouse-derived antibody targeting a BCMA antigen is obtained through screening test, and a plurality of sequences of a humanized scFv antibody targeting a BCMA antigen after optimizing the sequence of the mouse-derived antibody. A CAR containing the antibody is constructed and is transduced into human NK cells to successfully prepare CAR-NK cells, which exhibit good therapeutic effects in both in vivo and in vitro experiments. The CAR-NK cells obtained by humanized antibody are higher in stability of positive rate and in killing ability than the mouse-derived antibody.

Specifically, the present disclosure provides the following technical solutions.

In a first aspect, the present disclosure provides an antibody or an antigen binding fragment thereof targeting a BCMA, the antibody containing a variable light chain and a variable heavy chain,

the variable light chain containing a light chain CDR1, a light chain CDR2, and a light chain CDR3, the light chain CDR1 having a sequence as shown in SEQ ID NO. 1, the light chain CDR2 having an amino acid sequence as shown in SEQ ID NO. 2, and the light chain CDR3 being as shown in SEQ ID NO. 3 or SEQ ID NO. 4; and

the variable heavy chain containing a heavy chain CDR1, a heavy chain CDR2, and a heavy chain CDR3, the heavy chain CDR1 having a sequence as shown in SEQ NO. 5, the heavy chain CDR2 having an amino acid sequence as shown in SEQ ID NO. 6, and the heavy chain CDR3being as shown in SEQ ID NO. 7 or SEQ ID NO. 8.

Preferably, the antibody further includes a light chain FR1, a light chain FR2, a light chain FR3, a light chain FR4, a heavy chain FR1, a heavy chain FR2, a heavy chain FR3 and a heavy chain FR4.

Preferably, the light chain FR1 may be as shown in any one of SEQ ID NO. 9-10. Preferably, the light chain FR2 may be as shown in any one of SEQ ID NO. 11-12. Preferably, the light chain FR3 may be as shown in any one of SEQ ID NO. 13-14. Preferably, the light chain FR4 may be as shown in any one of SEQ ID NO. 15-16. Preferably, the heavy chain FR1 may be as shown in any one of SEQ ID NO. 17-18. Preferably, the heavy chain FR2 may be as shown in any one of SEQ ID NO. 19-21. Preferably, the heavy chain FR3 may be as shown in any one of SEQ ID NO. 22-24. Preferably, the heavy chain FR4 may be as shown in any one of SEQ ID NO. 25-26.

Preferably, the antibody has a link mode of the variable light chain-a connexon-the variable heavy chain.

More preferably, the antibody is humanized. Specifically, the humanized antibody is more stable in expression and stronger in killing target cells.

More preferably, the antibody has a complete sequence as shown in SEQ ID NO. 28-35.

The term “antigen binding fragment” used herein refers to an antibody fragment formed by a portion of the antibody, and contains one or more CDRs or any other antibody fragments bonded to an antigen but free of a complete natural antibody structure. For example, the antigen binding fragment includes, but is not limited to, bifunctional antibodies, Fab, Fab′, F(ab′)2, Fv fragments, disulfide bond-stabilized Fv fragments (dsFv), (dsFv)2, bispecific dsFv (dsFv-dsFv′), disulfide bond-stabilized bifunctional antibodies (ds bifunctional antibodies), single chain antibody molecules (scFv), scFv dimers (bivalent bifunctional antibodies), bispecific antibodies, multispecific antibodies, camelid single structural domain antibodies, nanobodies, structural domain antibodies, and bivalent structural domain antibodies. The antigen binding fragment is capable of binding to the same antigen that a parental antibody binds to.

More preferably, the antibody further contains a heavy chain constant region and a light chain constant region.

In another aspect, the present disclosure provides an antibody complex, the antibody complex being formed by linking a structural domain with a peptide bond to an amino terminal and/or a carboxyl terminal of the foregoing antibody or an antigen binding fragment thereof.

Preferably, the structural domain includes a hinge region, a transmembrane structural domain and/or a signal transduction structural domain.

Preferably, the signal transduction structural domain includes a co-stimulatory structural domain and/or an intracellular signal transduction structural domain.

Preferably, the hinge region includes one or a combination of a cluster of differentiation (CD) 8 hinge region, a CD28 hinge region, a CD4 hinge region, a CD5 hinge region, a CD134 hinge region, a CD137 hinge region, and an inducible co-stimulator (ICOS) hinge region.

Preferably, the hinge chain is the CD8 hinge chain, having an amino acid sequence as shown in SEQ ID NO. 45.

Preferably, the transmembrane structural domain includes a transmembrane structural domain of a protein, the protein including proteins expressed by a 2B4 gene, an a, B or (chain of a T cell receptor, CD28, CD38, CD45, CD4, CD5, CD8, CD9, CD16, CD22, CD33, CD37, CD64, CD80, CD86, CD123, CD134, CD137 and CD154.

Preferably, the transmembrane structural domain is the CD8 transmembrane structural domain, having an amino acid sequence as shown in SEQ ID NO. 46.

Preferably, the co-stimulatory structural domain includes a functional signal transduction structural domain of 4-1BB, 2B4, CD3ζ, OX40, CD2, CD27, CD28, CDS, ICAM-1, LFA-1 (CD11a/CD18), ICOS (CD278) and 4-1BB (CD137).

Preferably, the co-stimulatory structural domain is the 4-1BB co-stimulatory structural domain, having an amino acid sequence as shown in SEQ ID NO. 47.

Preferably, the intracellular signal transduction structural domain includes a signal transduction region formed arbitrarily by one or more proteins of CD3-ζ, FcεRIγ, FcRγ, FcRβ, CD3γ, CD3δ, CD3ε, CD5, CD22, CD79a, CD79b and CD66d.

Preferably, the intracellular signal transduction structural domain has an amino acid sequence as shown in SEQ ID NO. 48.

More preferably, the antibody complex is CAR.

Preferably, the structural domain further includes a signal peptide.

Preferably, a CD8 signal peptide is selected for testing in the present disclosure.

The CAR used herein, a receptor synthesized artificially, can direct immune cells to specifically trace, identify and remove tumor cells expressing related targeting ligands. The CAR is typically formed by an extracellular binding domain that can identify tumor-associated antigens (typically being scFv fragments from an antigen binding region of a monoclonal antibody), a hinge domain, a transmembrane domain, and an intracellular signal structural domain, and is of highly targeted.

In another aspect, the present disclosure provides a nucleic acid molecule encoding the foregoing antibody.

Preferably, the nucleic acid molecule is as shown in SEQ ID NO. 37-44.

In another aspect, the present disclosure provides a nucleic acid molecule encoding the foregoing antibody complex.

In another aspect, the present disclosure provides a vector, the vector containing the forgoing nucleic acid molecule encoding an antibody and/or a deoxyribonucleic acid (DNA) molecule of the forgoing polypeptide complex, or, the vector expressing the forgoing antibody or an antigen binding fragment thereof targeting a BCMA, or, an antibody complex.

Preferably, the vector is an expression vector.

Preferably, the vector is a virus expression vector.

Preferably, the virus expression vector includes a lentiviral vector, an adenoviral vector,

an adeno-associated virus expression vector or other types of viral vectors.

In another aspect, the present disclosure provides a host cell, the host cell containing one or more of the foregoing antibody, the foregoing polypeptide complex, a DNA molecule encoding the foregoing antibody, a DNA molecule of the foregoing polypeptide complex, and the foregoing vector.

Preferably, the host cell includes prokaryotic cells and eukaryotic cells.

Preferably, the prokaryotic cells are bacterial cells such as Agrobacterium and Escherichia coli.

Preferably, the bacterial cell includes Gram-negative microorganisms and Gram-positive microorganisms.

Preferably, the eukaryotic cells are fungal cells, i.e. yeast cells.

Preferably, the eukaryotic cells are animal cells, insect cells, and plant cells.

Preferably, the animal cell includes human-derived cells or non-human-derived cells. Preferably, the human-derived cells are immune cells.

Preferably, the immune cell includes one or more of T cells, B cells, K cells, and NK cells. Preferably, the immune cells are the NK cells or the T cells.

Preferably, the immune cells are autologous or xenogenous.

Preferably, the immune cell is derived from peripheral blood (PB), umbilical cord blood (UCB), human embryonic stem cells (HESC), induced pluripotent stem cells (IPSC) or a commercialized cell line.

In another aspect, the present disclosure provides an antibody conjugate, the antibody conjugate containing the forgoing antibody, and further containing a conjugated portion selected from the following group: a detectable marker, a drug, a toxin, a cytokine or an enzyme.

In another aspect, the present disclosure provides a drug composition for treating BCMA-related diseases, the drug composition containing one or more of the forgoing antibody or an antigen binding fragment thereof, the forgoing antibody complex, a nucleic acid molecule encoding the forgoing antibody, a nucleic acid molecule encoding the forgoing antibody complex, a vector, a host cell, and an antibody conjugate; and/or a pharmaceutically acceptable vector.

Preferably, the drug composition may further contain other active ingredients, such as sunitinib (sutent), iressa (gefitinib tablets), tarceva (erlotinib), herceptin (trastuzumab), avastin (bevacizumab), arsenic trioxide, trans-retinoic acid, velcade (bortezomib), temodar, erbitux (cetuximab), dasatinib (sprycel), sorafenib (nexavar), vectibix (panitumumab), TS-1, and ixempra (ixabepilone).

Preferably, the drug composition may further contain a pharmaceutically acceptable excipient.

Preferably, the pharmaceutically acceptable excipient includes any one or a combination of at least two of a diluent, a vehicle, a filling agent, a binding agent, a wetting agent, a disintegrant, an emulsifier, a cosolvent, a solubilizer, an osmotic pressure regulator, a surfactant, a coating material, a staining agent, a pH regulator, an antioxidant, a bacteriostatic agent or a buffering agent.

Preferably, the drug combined composition is in the form of capsules, tablets, pills, powders, granules, emulsions, solutions, suspensions, syrups, or tinctures.

In another aspect, the present disclosure provides a method for detection of BCMA, the method including a step of contacting the forgoing antibody or an antigen binding fragment thereof with a sample to be detected.

Preferably, the antibody or the antigen biding fragment thereof is an antibody having a detectable marker.

Preferably, the detectable marker includes an enzyme, a fluorescent material, a luminescent material, a radioactive material, a positron emission metal, and a non-radioactive paramagnetic metal ion.

Preferably, the method further includes a step of detecting a reaction between the sample to be detected and the antibody after being bonded.

Preferably, the sample to be detected includes a physiological body fluid, the physiological body fluid including blood, a serum, plasma, saliva, an eye secretion, a cerebrospinal fluid, pus, an exudate, breast milk, sweat, a tear, an car effluent, a sputum, lymph, urine or feces; and the sample to be detected further includes a tissue, the tissue including a lung, a spleen and a kidney.

Preferably, the sample to be detected contains B cells.

Preferably, the detection is for non-diagnostic purposes.

In another aspect, the present disclosure provides a treatment method for cancers, the method including administering the foregoing antibody or an antigen binding fragment thereof, the foregoing polypeptide complex, a nucleic acid molecule encoding the foregoing antibody, a nucleic acid molecule encoding the foregoing antibody complex, the foregoing vector, the foregoing host cell, or the foregoing drug composition to a subject.

Preferably, a mode of the administration includes enteral or non-enteral administration, such as oral, intravenous, intramuscular, subcutaneous, nasal, oral mucosal, ocular, pulmonary and respiratory, dermal, vaginal, or rectal administration.

Most preferably, the cancer is MM.

Preferably, the treatment method may be combined with other therapy approaches including chemotherapy, radiotherapy, immunotherapy, gene therapy, and surgery.

In another aspect, the present disclosure provides an application of any one or more of the foregoing antibody or an antigen binding fragment thereof, the foregoing polypeptide complex, a nucleic acid molecule encoding the foregoing antibody, a nucleic acid molecule encoding the foregoing antibody complex, the foregoing vector, the foregoing host cell, or the foregoing drug composition in combination with BCMA.

In another aspect, the present disclosure provides an application of any one or more of the foregoing antibody or an antigen binding fragment thereof, the foregoing polypeptide complex, a nucleic acid molecule encoding the foregoing antibody, a nucleic acid molecules encoding the foregoing antibody complex, the foregoing vector, the foregoing host cell, or the foregoing drug composition in the preparation of a drug for treating cancers or immune cell-mediated immune diseases expressing BCMA.

Preferably, the cancer includes a plasma cell malignant tumor or a B-cell malignant tumor.

Preferably, the cancer includes leukemia, lymphoma or myeloma.

Preferably, the cancer includes cancers associated with high BCMA expression.

Most preferably, the cancer includes MM, Hodgkin's lymphoma, leukemia, glioblastoma, non-Hodgkin's lymphoma (NHL), myelodysplastic syndrome (MDS), myeloproliferative syndrome (MPS), monoclonal gammopathy of undetermined significance (MGUS), solitary plasmacytoma, Waldenstrom macroglobulinemia, AL amyloidosis, plasma cell leukemia, and Burkett's lymphoma.

Most preferably, the cancer is MM.

Preferably, the immune cells are B cells.

Preferably, the immune disease includes rheumatoid arthritis, systemic lupus erythematosus (SLE), type I diabetes mellitus, asthma, atopic dermatitis, allergic rhinitis, thrombocytopenia purpura, multiple sclerosis, psoriasis, Sjogren's syndrome, Hashimoto's thyroiditis, Grave's diseases, primary biliary sclerosis, Wegener's granulomatosis, tuberculosis, and graft versus host diseases.

### BRIEF DESCRIPTION OF THE DRAWINGS

FIG. 1A is a graph showing the affinity of a mouse-derived antibody to BCMA in an H929 blank group;

FIG. 1B is a graph showing the affinity of the mouse-derived antibody to BCMA in an H929+PE fluorescence group;

FIG. 1C is a graph showing the affinity of the mouse-derived antibody to BCMA in a negative control group;

FIG. 1D is a graph showing the affinity of the mouse-derived antibody to BCMA in a positive control group; and

FIG. 1E is a graph showing the affinity of the mouse-derived antibody to BCMA.

FIG. 2A is a graph showing the expression of BCMA-CAR in NK cells in a negative control group;

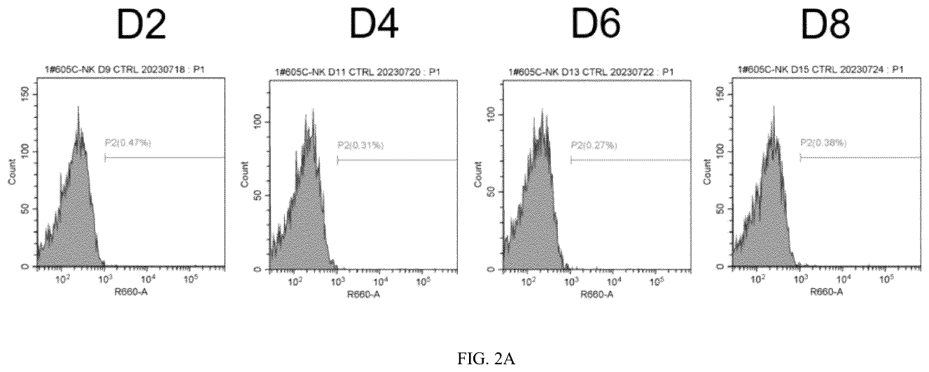

FIG. 2B is a graph showing the expression of mouse-derived BCMA-CAR in NK cells;

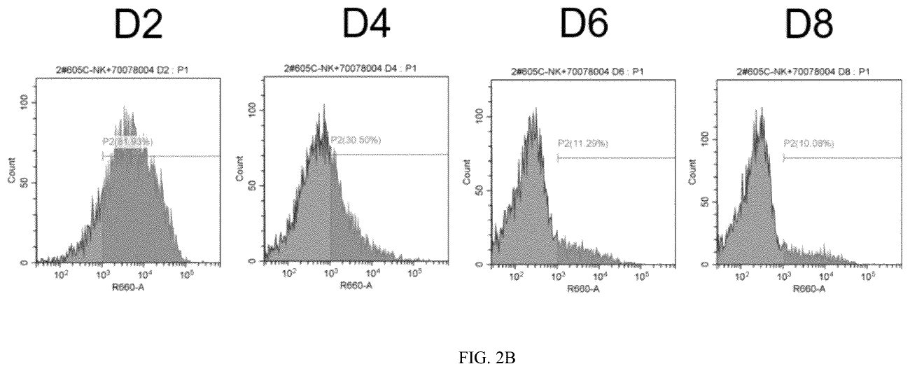

FIG. 2C is a graph showing the expression of humanized BCMA-CAR-V0 in NK cells;

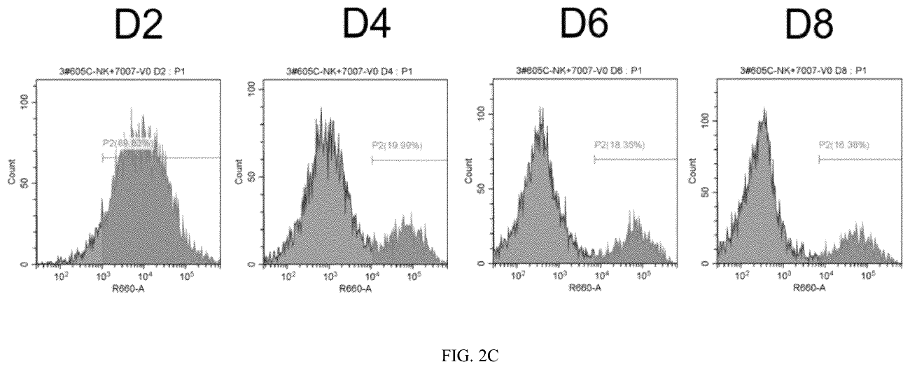

FIG. 2D is a graph showing the expression of humanized BCMA-CAR-V1 in NK cells;

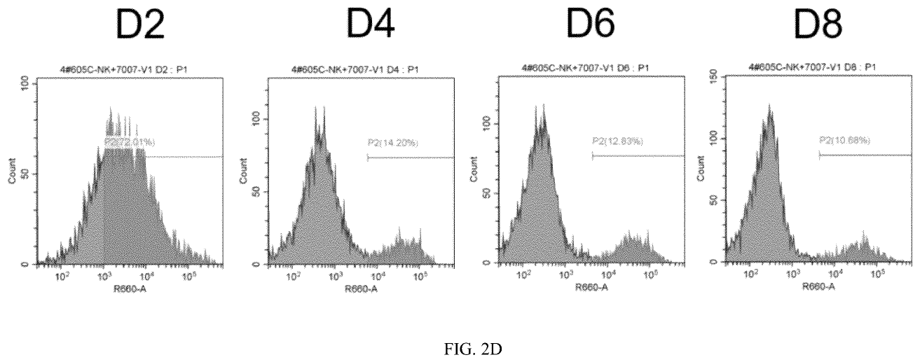

FIG. 2E is a graph showing the expression of humanized BCMA-CAR-V2 in NK cells;

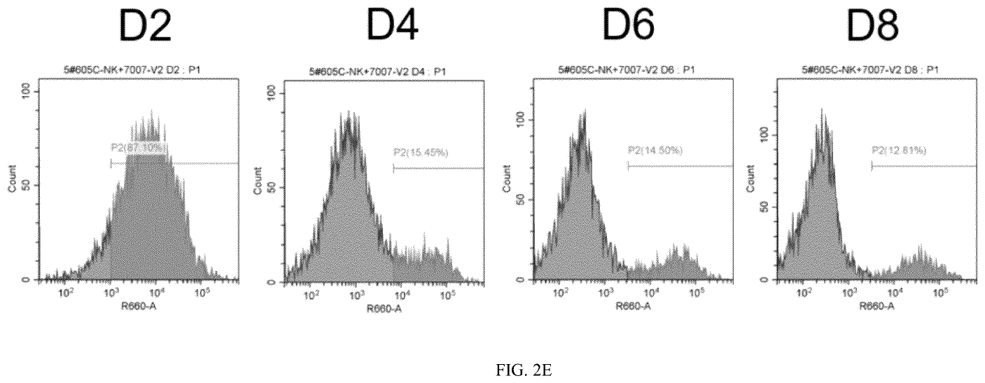

FIG. 2F is a graph showing the expression of humanized BCMA-CAR-V3 in NK cells;

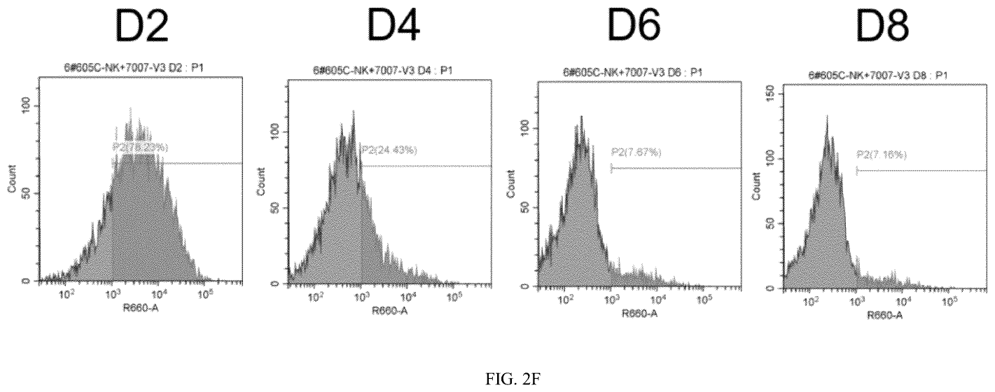

FIG. 2G is a graph showing the expression of humanized BCMA-CAR-V4 in NK cells;

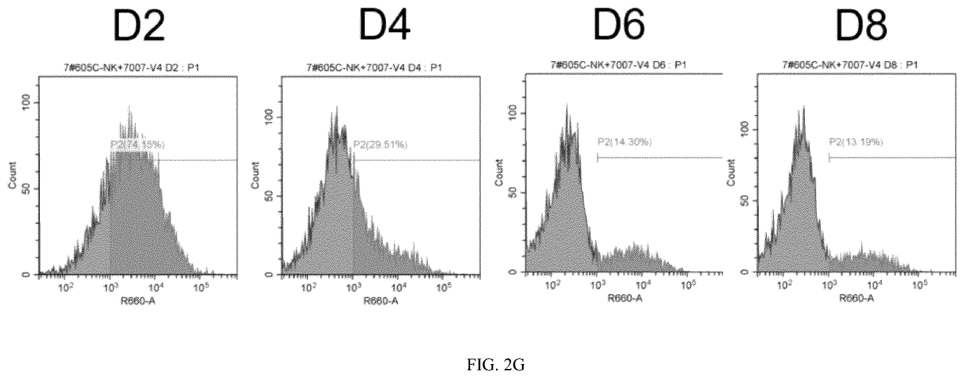

FIG. 2H is a graph showing the expression of humanized BCMA-CAR-V6 in NK cells;

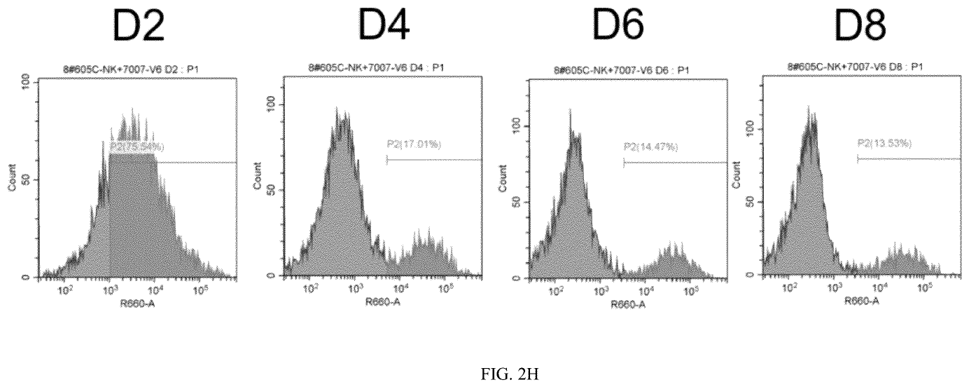

FIG. 2I is a graph showing the expression of humanized BCMA-CAR-V7 in NK cells; and

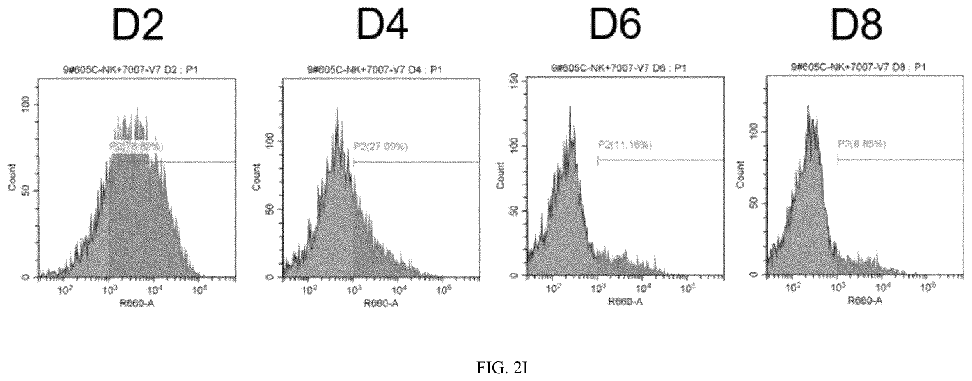

FIG. 2J is a graph showing the expression of humanized BCMA-CAR-V8 in NK cells.

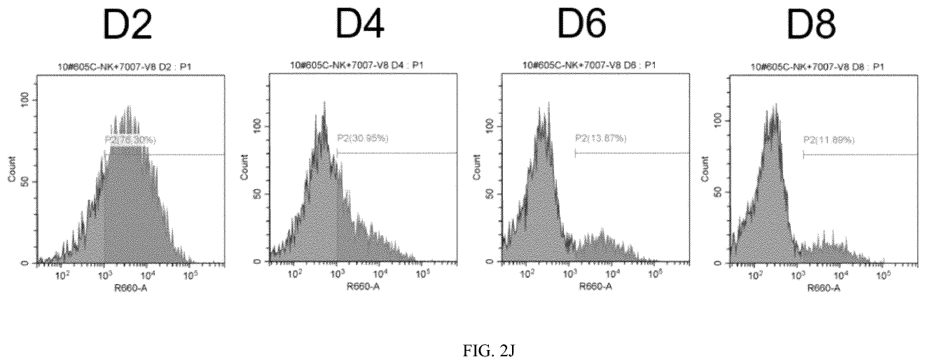

FIG. 3A is a graph showing the killing ability of BCMA-CAR in a negative control group;

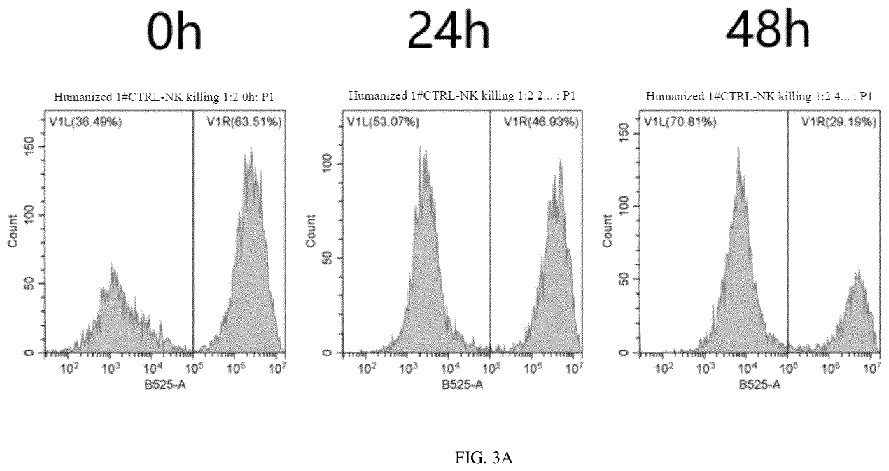

FIG. 3B is a graph showing the killing ability of mouse-derived BCMA-CAR;

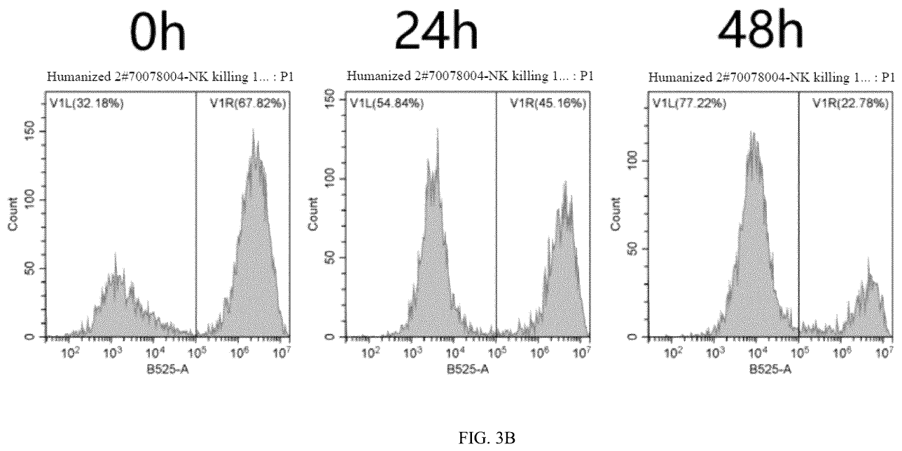

FIG. 3C is a graph showing the killing ability of humanized BCMA-CAR-V0;

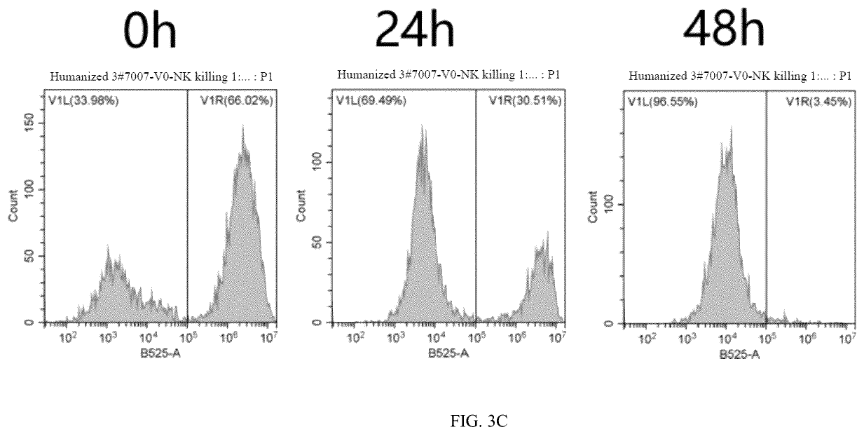

FIG. 3D is a graph showing the killing ability of humanized BCMA-CAR-V1;

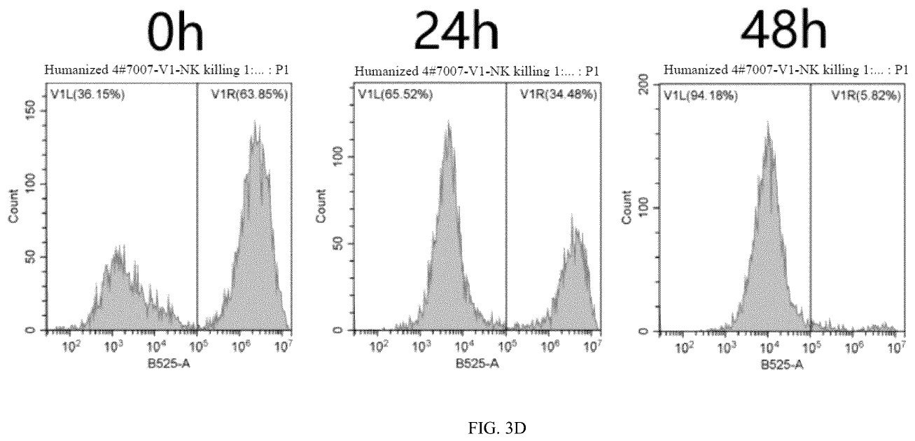

FIG. 3E is a graph showing the killing ability of humanized BCMA-CAR-V2;

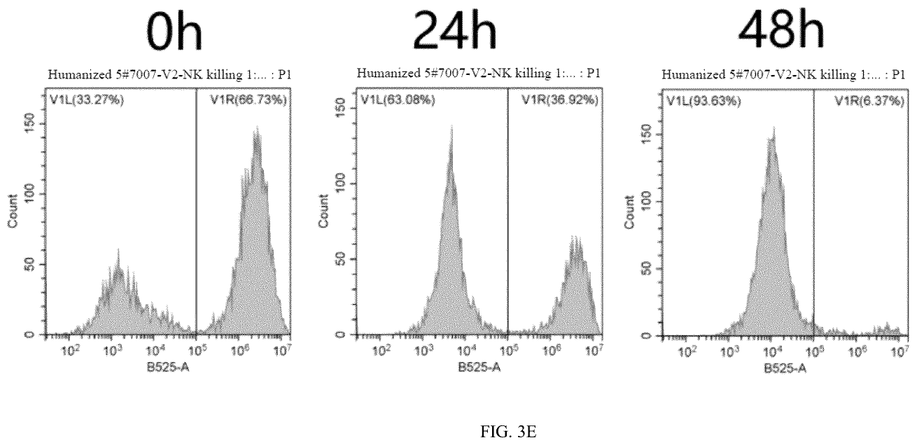

FIG. 3F is a graph showing the killing ability of humanized BCMA-CAR-V3;

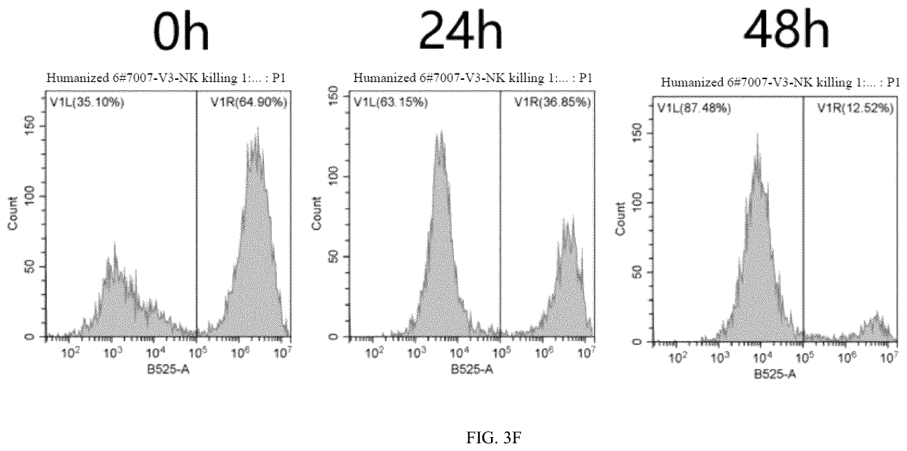

FIG. 3G is a graph showing the killing ability of humanized BCMA-CAR-V4;

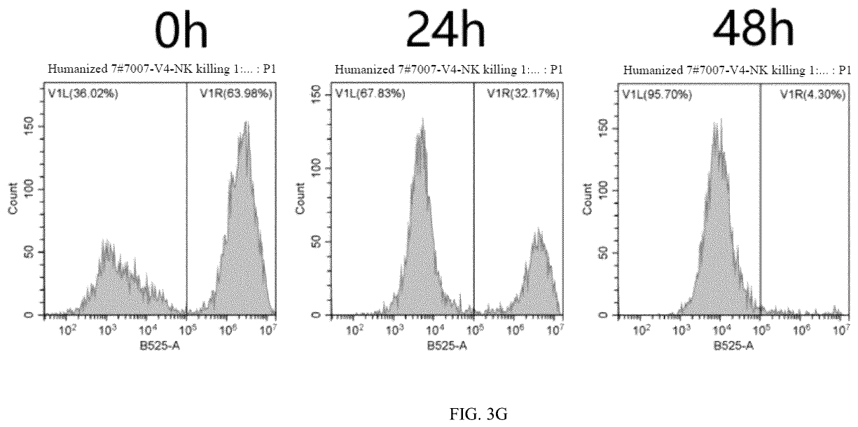

FIG. 3H is a graph showing the killing ability of humanized BCMA-CAR-V6;

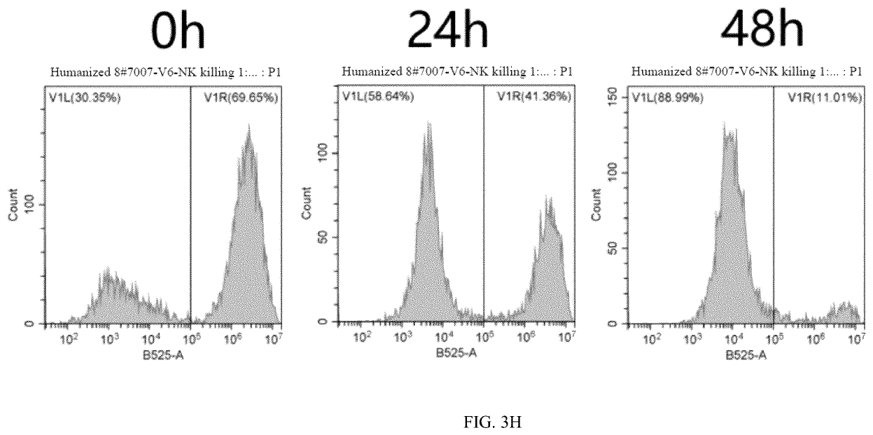

FIG. 3I is a graph showing the killing ability of humanized BCMA-CAR-V7; and

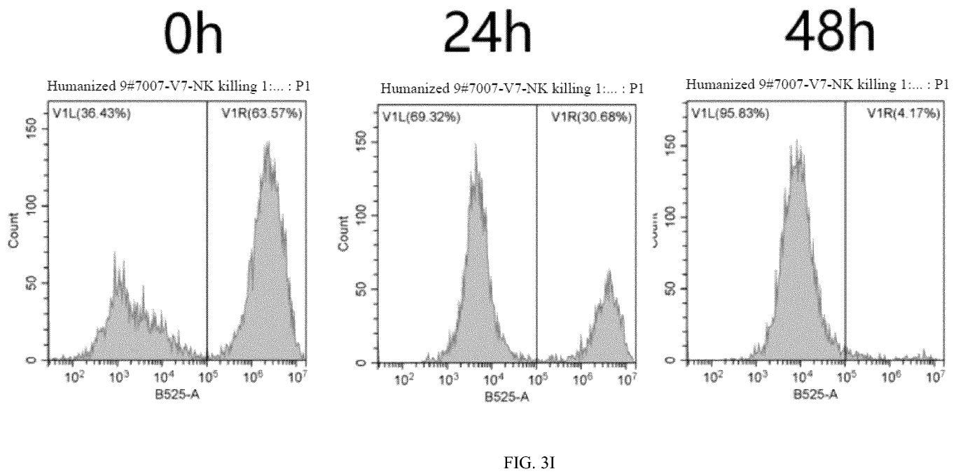

FIG. 3J is a graph showing the killing ability of humanized BCMA-CAR-V8.

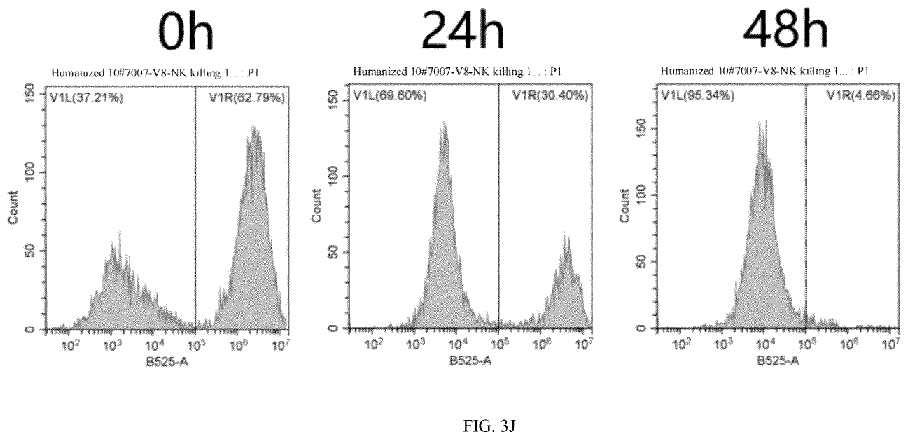

### DETAILED DESCRIPTION

The present disclosure is further described below by reference to the embodiments, and what described in the following is merely the preferred embodiments of the present disclosure, rather than a limitation to the present disclosure in any other form. The above disclosed technical contents can be altered to equivalent embodiments by any skilled familiar with the field, and without departing from the content of the solutions of the present disclosure, any simple modifications or equivalent changes to the following embodiments based on the technical substance of the present disclosure fall within the scope of protection of the present disclosure.

### Embodiment 1, Determination of the Affinity of Mouse-Derived Antibody to Myeloma Cells

A mouse-derived antibody was obtained and the affinity of the antibody to myeloma cells

was detected. The mouse-derived antibody had an amino acid sequence as shown in SEQ ID NO. 27 and a nucleotide sequence as shown in SEQ ID NO. 36.

### Determination Method

1. H929 myeloma cells were cultured, and then subjected to cell counting. 5×10⁵ cells were taken and placed in a 1.5 mL Eppendorf (EP) tube for centrifuging at 1000 rpm for 5 min, and supernatant was removed.

2. 500 μL of phosphate buffered saline (PBS) was added to resuspend the cells, and the resuspended cells were centrifuged at 1000 rpm for 5 min, and supernatant was removed.

3. Supernatant from blank cell culture and supernatant produced from antibody were separately used to resuspend cells to be tested. The resuspended cells were protected from light for reacting at 4° C. for 30 min, and then centrifuged at 1000 rpm for 5 min, and supernatant was removed.

4. 500 μL of PBS was added to resuspend the cells, and the resuspended cells were centrifuged at 1000 rpm for 5 min, and supernatant was removed.

5. 100 μL of secondary antibody (PE anti-human IgG Fc Antibody, diluted by 1:600 with PBS) was added to resuspend the cells. The resuspended cells were protected from light for reacting at 4° C. for 30 min, and then centrifuged at 1000 rpm for 5 min, and supernatant was removed.

6. 500 μL of PBS was added to resuspend the cells, and the resuspended cells were centrifuged at 1000 rpm for 5 min, and supernatant was removed.

7. 400 μL of PBS was added to resuspend the cells, and the resuspended cells were detected by a machine.

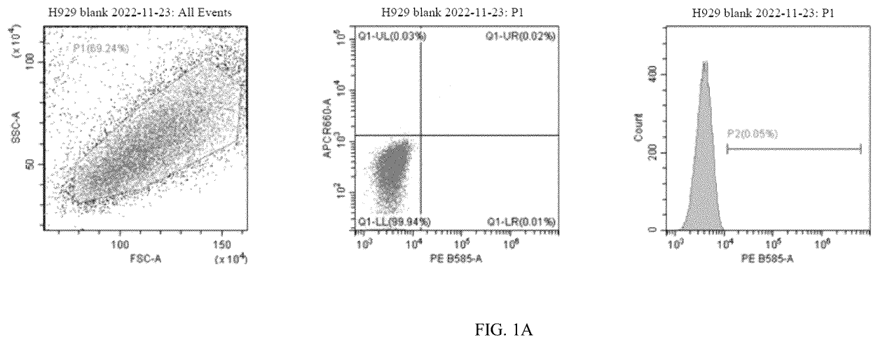

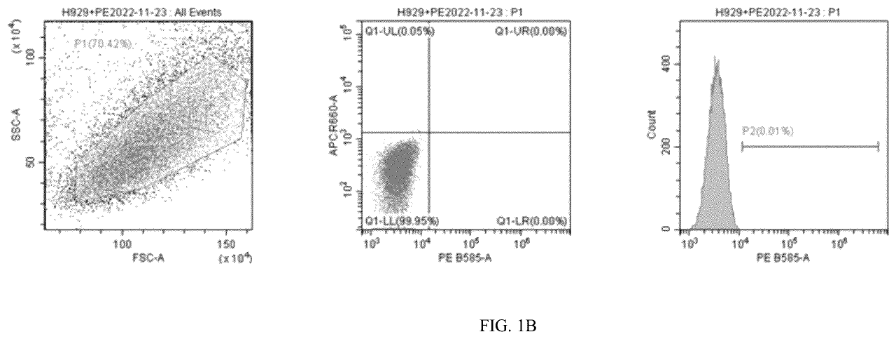

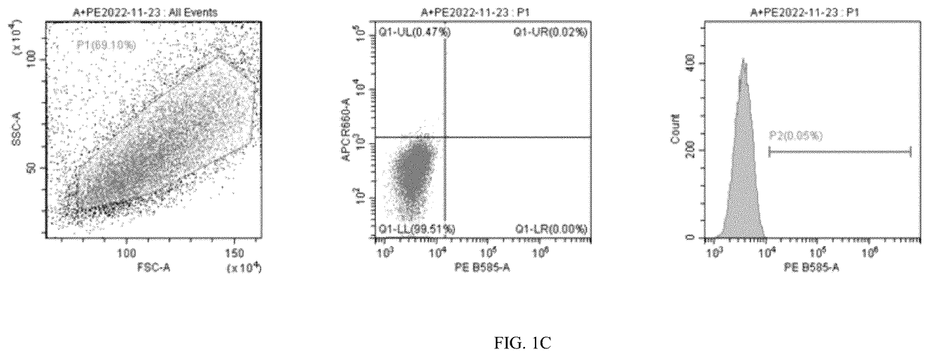

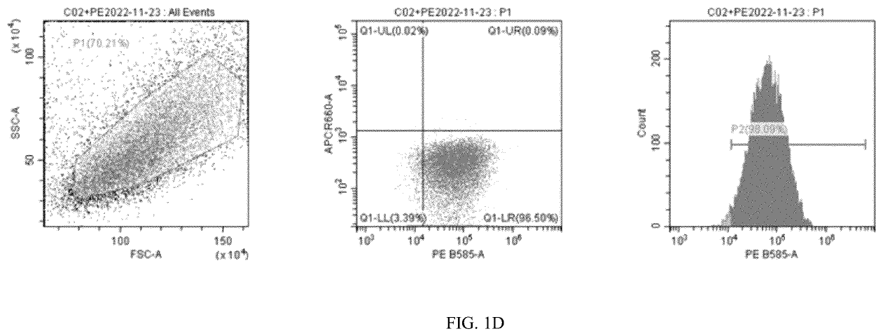

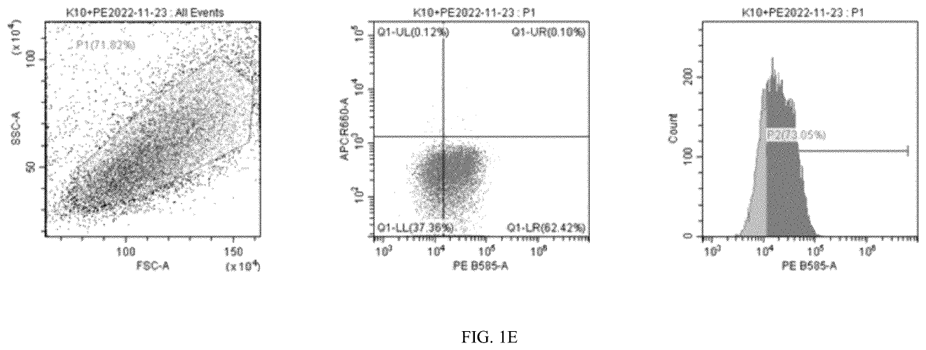

The results prove that the mouse-derived BCMA antibody provided in the present disclosure has good affinity to H929 cells. The positive rate of PE-fluorescence labeled H929 cells is shown in Table 1, and the detection results are shown in FIG. 1.

TABLE 1
PE positive rate statistics

| | Allophycocyanin (APC) |
|---|---|
| Group | fluorescent positive rate |
| H929 blank | 0.05% |
| H929 + PE | 0.01% |
| Negative control | 0.05% |
| Positive control | 98.09% |
| BCMA-scFv candidate | 73.05% |

### Embodiment 2, Detection of Transduction Rate of CAR Constructed by Humanized Antibody to Cells

### Acquisition of Humanized Antibody

Mouse-derived antibodies were humanized. Specifically, a sequence of a light chain CDR1 of each of the humanized antibodies was as shown in SEQ ID NO. 1, an amino acid sequence of a light chain CDR2 was as shown in SEQ ID NO. 2, and a light chain CDR3 was as shown in SEQ ID NO. 3 or SEQ ID NO. 4. A sequence of a heavy chain CDR1 of each of the humanized antibodies was as shown in SEQ ID NO. 5, an amino acid sequence of a heavy chain CDR2 was as shown in SEQ ID NO. 6, and a heavy chain CDR3 was as shown in SEQ ID NO. 7 or SEQ ID NO. 8.

In the case that the above CDR (also known as hypervariable region) remains unchanged, different FRs were also tested, proving that the antibodies formed by different FRs were functional in the case that the above CDR remains unchanged. Specifically,

a light chain FR1 could be shown as any one of SEQ ID NO. 9-10, a light chain FR2 could be shown as any one of SEQ ID NO. 11-12, a light chain FR3 could be shown as any one of SEQ ID NO. 13-14, and a light chain FR4 could be shown as any one of SEQ ID NO. 15-16. A heavy chain FR1 could be shown as any one of SEQ ID NO. 17-18, a heavy chain FR2 could be shown as any one of SEQ ID NO. 19-21, a heavy chain FR3 could be shown as any one of SEQ ID NO. 22-24, and a heavy chain FR4 could be shown as any one of SEQ ID NO. 25-26.

Humanized antibodies V0, V1, V2, V3, V4, V6, V7, and V8 were further prepared for testing. Specifically, amino acid sequences of the humanized antibodies V0, V1, V2, V3, V4, V6, V7, and V8 were sequentially shown as SEQ ID NO. 28-35, and coding nucleic acid sequences were sequentially shown as SEQ ID NO. 37-44.

### Construction of CAR

A CD8 signal peptide (shown as SEQ ID NO. 45) was ligated in front of the above mouse-derived and humanized antibodies, and a CD8 hinge region +a transmembrane domain (shown as SEQ ID NO. 46), a 4-1BB co-stimulatory structural domain (shown as SEQ ID NO. 47), and an intracellular signal transduction structural domain (shown as SEQ ID NO. 48) were ligated after the mouse-derived antibody, and BCMA-CAR-V0/V1/V2/V3/V4/V6/V7/V8 was obtained for transduction.

That is, the function of CAR was verified in the present disclosure through the following structure: CD8 signal peptide-antibody-CD8 hinge region-CD8 transmembrane domain-4-1BB co-stimulatory structural domain-intracellular signal transduction structural domain.

### Preparation of CAR-NK Cells

A third-generation lentiviral packaging system and a four-plasmid system were employed to package three plasmids: pRSV-Rev, pMDLg/pRRE, and pMD2.G, and shuttle pLVX-puro, and HEK293T cells served as virus production cells, at a mixing ratio of pLenti-puro:pRSV-Rev:pMDLg/pRRE:pMD2.G of 2:1:1:1. A lipidosome mirusTransIT-293 (mir2700) acted as a transfection reagent, with a lipidosome-to-DNA ratio of 3:1. After the transfection, supernatant was collected in three days and centrifuged at a high speed of 40000 g for 6 h.

Viruses at an infection concentration of 10 MOI were directly added to a culture medium of NK cells, and at the same time, 0.8 μg/mL of polybrene was added, followed by incubating for 6 h. An NK medium containing viruses was removed, and a fresh NK medium was added for continuous culture. The CAR positive rate was determined by a flow cytometry at different time points.

### Results Analysis

TABLE 2
Flow cytometry results of BCMA-CAR expression and stability

| Numerical | | | | | |
|---|---|---|---|---|---|
| order | Group | Day 2 | Day 4 | Day 6 | Day 8 |
| 1 | Negative control | 0.47% | 0.31% | 0.27% | 0.38% |
| 2 | Mouse-derived | 81.93% | 30.50% | 11.29% | 10.08% |
| | BCMA-CAR | | | | |
| 3 | Humanized BCMA- | 89.83% | 19.99% | 18.35% | 16.38% |
| | CAR-V0 | | | | |
| 4 | Humanized BCMA- | 72.01% | 14.20% | 12.83% | 10.68% |
| | CAR-V1 | | | | |
| 5 | Humanized BCMA- | 87.10% | 15.45% | 14.50% | 12.81% |
| | CAR-V2 | | | | |
| 6 | Humanized BCMA- | 78.23% | 24.43% | 7.67% | 7.16% |
| | CAR-V3 | | | | |
| 7 | Humanized BCMA- | 74.15% | 29.51% | 14.30% | 13.19% |
| | CAR-V4 | | | | |
| 8 | Humanized BCMA- | 75.54% | 17.01% | 14.47% | 13.53% |
| | CAR-V6 | | | | |
| 9 | Humanized BCMA- | 76.82% | 27.09% | 11.16% | 8.85% |
| | CAR-V7 | | | | |
| 10 | Humanized BCMA- | 76.30% | 30.95% | 13.87% | 11.89% |
| | CAR-V8 | | | | |

The flow cytometry results of humanized BCMA-CAR expression and stability are shown in Table 2, showing that BCMA-CAR-V0/V1/V2/V3/V4/V6/V7/V8 can transduce NK cells well, and the transduction rate of each group can reach between 70%-90% on Day 2.

From Day 4, the BCMA-CAR expression tends to be stable, and the difference in the stability of expression between groups is shown. The BCMA-CAR expression in the mouse-derived group decreases significantly to 30.5%, with inconspicuous clustering. The stability of expression in each humanized group is different. In the four groups of BCMA-CAR-V0/V1/V2/V6, the stability of expression is better, with conspicuous cell clustering, and the proportions of CAR-NK cells are 19.99%, 14.2%, 15.45% and 17.01%, respectively. The histogram shows an obvious BCMA-CAR-NK cell cluster forming on the right side.

On Day 6-Day 8, the BCMA-CAR expression in the mouse-derived group further decreases to 11.29%, with inconspicuous CAR-NK cell clustering, while the stability of expression in the five humanized groups of BCMA-CAR-V0/V1/V2/V4/V6 is better, with conspicuous cell clustering and CAR-NK cells accounting for 18.35%, 12.83%, 14.5%, 14.3%, and 14.47%, respectively.

Compared with mouse-derived BCMA-CAR, the humanized BCMA-CAR has a more stable and uniform expression on an NK cell membrane, and can form a clearer group of NK cell clusters (V0/V1/V2/V4/V6) expressing CAR. In the histogram, BCMA-CAR-NK cells are shown as a small cell peak on the right side, and the more conspicuous of this peak is, the more uniform and stable of the CAR expression is.

### Embodiment 3, Cell Killing Experiment

BCMA-CAR-NK cells in each group were expanded, cultured, and counted.

A target cell line with green fluorescence: H929-GFP cell was cultured, and counted.

The BCMA-CAR-NK cells and the H929-GFP cells were co-cultured at an effector-target ratio of 1:2.

The proportion of H929-GFP cells in a co-culture system was detected by a flow cytometry at different time points (24 h, and 48 h).

### Results Analysis

TABLE 3
Detection results of cell killing at effector-target ratio of 1:2

| | | | | | 24 h | 48 h |
|---|---|---|---|---|---|---|
| Numerical | | | | | killing | killing |
| order | Group | 0 h | 24 h | 48 h | ratio | ratio |
| 1 | Negative control without virus | 63.51% | 46.93% | 29.19% | 26.11% | 54.04% |
| 2 | Mouse-derived BCMA-CAR | 67.82% | 45.16% | 22.78% | 33.41% | 66.41% |
| 3 | Humanized BCMA-CAR-V0 | 66.02% | 30.51% | 3.45% | 53.79% | 94.77% |
| 4 | Humanized BCMA-CAR-V1 | 63.85% | 34.48% | 5.82% | 46.00% | 90.88% |
| 5 | Humanized BCMA-CAR-V2 | 66.73% | 36.92% | 6.37% | 44.67% | 90.45% |
| 6 | Humanized BCMA-CAR-V3 | 64.90% | 36.85% | 12.52% | 43.22% | 80.71% |
| 7 | Humanized BCMA-CAR-V4 | 63.98% | 32.17% | 4.30% | 49.72% | 93.28% |
| 8 | Humanized BCMA-CAR-V6 | 69.65% | 41.36% | 11.01% | 40.62% | 84.19% |
| 9 | Humanized BCMA-CAR-V7 | 63.57% | 30.68% | 4.17% | 51.74% | 93.44% |
| 10 | Humanized BCMA-CAR-V8 | 62.79% | 30.40% | 4.66% | 51.58% | 92.58% |

The detection results of cell killing at effector-target ratio of 1:2 are shown in Table 3. At 24 h, compared with the proportion of 45.16% of the H929-GFP cells accounting in the mouse-derived BCMA-CAR-NK group, the H929-GFP cells account less in each humanized BCMA-CAR-NK group. The H929-GFP cells in the four groups of BCMA-CAR-V0/V4/V7/V8 account for 30.5%, 32.17%, 30.68%, 30.4%, and 30.4%, respectively.

At 48 h, compared with the proportion of 22.78% of the H929-GFP cells in the mouse-derived BCMA-CAR-NK group, the proportion of the H929-GFP cells in each humanized BCMA-CAR-NK group is significantly decreased. The H929-GFP cells in the four groups of BCMA-CAR-V0/V4/V7/V8 account for 3.45%, 4.3%, 4.17%, and 4.66%, respectively. The histogram shows that, the lower or even disappearing of the small cell peak on the right side, the higher the killing ability.

Compared with the mouse-derived BCMA-CAR-NK cells, the humanized BCMA-CAR-NK cells have a better killing ability to basically remove the tumor cells at 48 h, and show different killing ability in the presence of different humanized modifications. On the whole, the humanized BCMA-CAR-NK cells have a better killing ability than that of the mouse-derived BCMA-CAR-NK cells.

1. An antibody or an antigen binding fragment thereof targeting a B cell maturation antigen (BCMA), the antibody containing a variable light chain and a variable heavy chain,
the variable light chain containing a light chain complementarity-determining region (CDR) 1, a light chain CDR2, and a light chain CDR3, the light chain CDR1 having a sequence as shown in SEQ ID NO. 1, the light chain CDR2 having an amino acid sequence as shown in SEQ ID NO. 2, and the light chain CDR3 being as shown in SEQ ID NO. 3 or SEQ ID NO. 4; and
the variable heavy chain containing a heavy chain CDR1, a heavy chain CDR2, and a heavy chain CDR3, the heavy chain CDR1 having a sequence as shown in SEQ NO. 5, the heavy chain CDR2 having an amino acid sequence as shown in SEQ ID NO. 6, and the heavy chain CDR3 being as shown in SEQ ID NO. 7 or SEQ ID NO. 8.

2. The antibody or an antigen binding fragment thereof according to claim 1, wherein the antibody has a link mode of the variable light chain—a connexon—the variable heavy chain.

3. The antibody or an antigen binding fragment thereof according to claim 1, wherein the antibody is humanized.

4. The antibody or an antigen binding fragment thereof according to claim 1, wherein the antibody has a sequence as shown in any one of SEQ ID NO. 28-35.

5. The antibody or an antigen binding fragment thereof according to claim 1, wherein the antibody further contains a heavy chain constant region and a light chain constant region.

6. An antibody complex, formed by ligating the antibody or an antigen binding fragment thereof according to claim 1 to a cluster of differentiation (CD) 8 hinge region, a CD8 transmembrane domain, a 4-1BB co-stimulatory structural domain, and an intracellular signal transduction structural domain, the intracellular signal transduction structural domain having an amino acid sequence as shown in SEQ ID NO. 48.

7. The antibody complex according to claim 6, wherein the antibody complex further comprises a signal peptide.

8. The antibody complex according to claim 7, wherein the signal peptide is a CD8 signal peptide.

9. A nucleic acid molecule encoding the antibody or an antigen binding fragment thereof according to claim 1.

10. The nucleic acid molecule according to claim 9, wherein the nucleic acid molecule has a nucleic acid sequence as shown in any one of SEQ ID NO. 37-44.

11. A nucleic acid molecule encoding the antibody complex according to claim 6.

12. A vector, containing the nucleic acid molecule according to claim 9.

13. The vector according to claim 12, wherein the vector is an expression vector.

14. The vector according to claim 12, wherein the vector is a virus expression vector.

15. The vector according to claim 12, wherein the virus expression vector is selected from a lentiviral vector, an adenoviral vector, and an adeno-associated virus expression vector.

16. A vector, expressing the antibody or an antigen binding fragment thereof according to claim 1.

17. The vector according to claim 16, wherein the vector is an expression vector.

18. The vector according to claim 16, wherein the vector is a virus expression vector.

19. The vector according to claim 16, wherein the virus expression vector is selected from a lentiviral vector, an adenoviral vector, and an adeno-associated virus expression vector.

20. A vector, containing the nucleic acid molecule according to claim 11.

21. The vector according to claim 20, wherein the vector is an expression vector.

22. The vector according to claim 20, wherein the vector is a virus expression vector.

23. The vector according to claim 20, wherein the virus expression vector is selected from a lentiviral vector, an adenoviral vector, and an adeno-associated virus expression vector.

24. A vector, expressing the antibody complex according to claim 6.

25. The vector according to claim 24, wherein the vector is an expression vector.

26. The vector according to claim 24, wherein the vector is a virus expression vector.

27. The vector according to claim 24, wherein the virus expression vector is selected from a lentiviral vector, an adenoviral vector, and an adeno-associated virus expression vector.

28. A host cell, containing the antibody or an antigen binding fragment thereof according to claim 1.

29. The host cell according to claim 28, wherein the host cell is selected form prokaryotic cells and eukaryotic cells.

30. The host cell according to claim 29, wherein the eukaryotic cell is selected from animal cells, insect cells, and plant cells.

31. The host cell according to claim 30, wherein the animal cell is selected from human-derived cells or non-human-derived cells.

32. The host cell according to claim 31, wherein the human-derived cell is selected from immune cells or somatic cells.

33. The host cell according to claim 32, wherein the immune cell is selected from one or more of T cells, B cells, natural killer (NK) T cells, and NK cells.

34. The host cell according to claim 32, wherein the immune cell is the NK cell.

35. The host cell according to claim 34, wherein the NK cell is derived from peripheral blood (PB), umbilical cord blood (UCB), human embryonic stem cells (HESC), induced pluripotent stem cells (IPSC) or a cell line.

36. A host cell, containing the nucleic acid molecule according to claim 9.

37. The host cell according to claim 36, wherein the host cell is selected form prokaryotic cells and eukaryotic cells.

38. The host cell according to claim 37, wherein the eukaryotic cell is selected from animal cells, insect cells, and plant cells.

39. The host cell according to claim 38, wherein the animal cell is selected from human-derived cells or non-human-derived cells.

40. The host cell according to claim 39, wherein the human-derived cell is selected from immune cells or somatic cells.

41. The host cell according to claim 40, wherein the immune cell is selected from one or more of T cells, B cells, natural killer (NK) T cells, and NK cells.

42. The host cell according to claim 40, wherein the immune cell is the NK cell.

43. The host cell according to claim 42, wherein the NK cell is derived from peripheral blood (PB), umbilical cord blood (UCB), human embryonic stem cells (HESC), induced pluripotent stem cells (IPSC) or a cell line.

44. A host cell, containing the vector according to claim 12.

45. The host cell according to claim 44, wherein the host cell is selected form prokaryotic cells and eukaryotic cells.

46. The host cell according to claim 45, wherein the eukaryotic cell is selected from animal cells, insect cells, and plant cells.

47. The host cell according to claim 46, wherein the animal cell is selected from human-derived cells or non-human-derived cells.

48. The host cell according to claim 47, wherein the human-derived cell is selected from immune cells or somatic cells.

49. A host cell, containing the antibody complex according to claim 6.

50. A host cell, containing the nucleic acid molecule according to claim 11.

51. A host cell, containing the vector according to claim 20.

52. An antibody conjugate, containing the antibody or an antigen binding fragment thereof according to claim 1, and further containing a conjugated portion selected from the following group: a detectable marker, a drug, a toxin, a cytokine or an enzyme.

53. A drug composition for treating BCMA-related diseases, containing the antibody complex according to claim 6, the BCMA-related disease is multiple myeloma (MM), Burkitt's lymphoma, and plasma cell leukemia.

54. A drug composition for treating BCMA-related diseases, containing the nucleic acid molecule according to claim 11, the BCMA-related disease is multiple myeloma (MM), Burkitt's lymphoma, and plasma cell leukemia.

55. A drug composition for treating BCMA-related diseases, containing the vector according to claim 20, the BCMA-related disease is multiple myeloma (MM), Burkitt's lymphoma, and plasma cell leukemia.

56. A drug composition for treating BCMA-related diseases, containing the host cell according to claim 49, the BCMA-related disease is multiple myeloma (MM), Burkitt's lymphoma, and plasma cell leukemia.

57. The drug composition according to claim 56, wherein the drug composition further contains a pharmaceutically acceptable excipient.

58. The drug composition according to claim 56, wherein the pharmaceutically acceptable excipient is selected from any one or a combination of at least two of a diluent, a vehicle, a filling agent, a binding agent, a wetting agent, a disintegrant, an emulsifier, a cosolvent, a solubilizer, an osmotic pressure regulator, a surfactant, a coating material, a staining agent, a pH regulator, an antioxidant, a bacteriostatic agent or a buffering agent.

59. The drug composition according to claim 56, wherein the drug composition further contains any one or more of: sunitinib, iressa, tarceva, herceptin, avastin, arsenic trioxide, trans-retinoic acid, velcade, temodar, erbitux, dasatinib, sorafenib, vectibix, TS-1, and ixempra.

60. A drug composition for treating BCMA-related diseases, containing the antibody complex according to claim 6, the BCMA-related disease is myeloma.

61. A drug composition for treating BCMA-related diseases, containing the nucleic acid molecule according to claim 11, the BCMA-related disease is myeloma.

62. A drug composition for treating BCMA-related diseases, containing the vector according to claim 20 the BCMA-related disease is myeloma.

63. A drug composition for treating BCMA-related diseases, containing the host cell according to claim 49, the BCMA-related disease is myeloma.

64. The drug composition according to claim 63, wherein the drug composition further contains a pharmaceutically acceptable excipient.

65. The drug composition according to claim 63, wherein the pharmaceutically acceptable excipient is selected from any one or a combination of at least two of a diluent, a vehicle, a filling agent, a binding agent, a wetting agent, a disintegrant, an emulsifier, a cosolvent, a solubilizer, an osmotic pressure regulator, a surfactant, a coating material, a staining agent, a pH regulator, an antioxidant, a bacteriostatic agent or a buffering agent.

66. The drug composition according to claim 63, wherein the drug composition further contains any one or more of: sunitinib, iressa, tarceva, herceptin, avastin, arsenic trioxide, trans-retinoic acid, velcade, temodar, erbitux, dasatinib, sorafenib, vectibix, TS-1, and ixempra.

67. A method for detection of BCMA, comprising a step of contacting the antibody or an antigen binding fragment thereof according to claim 1 with a sample to be detected, the detection being for non-diagnostic purposes.

68. The method according to claim 67, wherein the antibody or an antigen biding fragment thereof is an antibody having a detectable marker.

69. The method according to claim 68, wherein the detectable marker is selected from an enzyme, a fluorescent material, a luminescent material, a radioactive material, a positron emission metal, and a non-radioactive paramagnetic metal ion.

70. The method according to claim 67, wherein the method further comprises a step of detecting a reaction between the sample to be detected and the antibody after being bonded.

71. The method according to claim 67, wherein the sample to be detected comprises a physiological body fluid, the physiological body fluid being selected from blood, a serum, plasma, saliva, an eye secretion, a cerebrospinal fluid, pus, an exudate, breast milk, sweat, a tear, an ear effluent, a sputum, lymph, urine or feces; and the sample to be detected further comprises a tissue, the tissue comprising a lung, a spleen and a kidney.

72. The method according to claim 67, wherein the sample to be detected contains B cells.

73. An application of the antibody complex according to claim 6 in the preparation of a drug for treating diseases, the disease being MM, Burkitt's lymphoma, or plasma cell leukemia.

74. An application of the nucleic acid molecule according to claim 11 in the preparation of a drug for treating diseases, the disease being MM, Burkitt's lymphoma, or plasma cell leukemia.

75. An application of the vector according to claim 20 in the preparation of a drug for treating diseases, the disease being MM, Burkitt's lymphoma, or plasma cell leukemia.

76. An application of the host cell according to claim 49 in the preparation of a drug for treating diseases, the disease being MM, Burkitt's lymphoma, or plasma cell leukemia.

77. An application of the antibody complex according to claim 6 in the preparation of a drug for treating diseases, the disease being myeloma.

78. An application of the nucleic acid molecule according to claim 11 in the preparation of a drug for treating diseases, the disease being myeloma.

79. An application of the vector according to claim 20 in the preparation of a drug for treating diseases, the disease being myeloma.

80. An application of the host cell according to claim 49 in the preparation of a drug for treating diseases, the disease being myeloma.
